(19) United States
(12) Patent Application Publication
(10) Pub. No.: US 2025/0025051 A1
(43) Pub. Date: Jan. 23, 2025

(54) NON-INVASIVE BLOOD GLUCOSE MONITORING DEVICE

(71) Applicant: Taiwan-Asia Semiconductor Corporation, Hsinchu City (TW)

(72) Inventors: Hsiu-Jung HUANG, Hsinchu City (TW); Sheng-Wei CHEN, Hsinchu City (TW)

(21) Appl. No.: 18/490,842

(22) Filed: Oct. 20, 2023

(30) Foreign Application Priority Data
Jul. 20, 2023 (TW) 112127228

(51) Int. Cl. A61B 5/00

(52) U.S. Cl. CPC A61B 5/0071; A61B 5/6832; A61B 2562/166

(57) ABSTRACT

The invention provides a non-invasive blood glucose monitoring device, which comprises a flexible circuit board, at least one blood glucose monitoring module and an adhesive layer. The at least one blood glucose monitoring module is arranged on the flexible circuit board, and each blood glucose monitoring module includes a barrier structure, a light emitting element, a light sensing element and a transparent cover. The barrier structure forms a first space and a second space isolated from each other; the light emitting element and the light sensing element are respectively located in the first space and the second space; the transparent cover is combined with the barrier structure to enclose the first space and the second space. The adhesive layer is arranged on the flexible circuit board and is located around the at least one blood glucose monitoring module.

### CROSS REFERENCE TO RELATED APPLICATION

This application claims the benefit of Taiwan Patent Application Serial No. 112127228 filed on Jul. 20, 2023. The entirety of each application is incorporated herein by reference.

### BACKGROUND OF THE INVENTION

### 1. Field of the Invention

The present invention relates to a blood glucose monitoring device, especially a non-invasive blood glucose monitoring device with a flexible circuit board as a substrate.

### 2. Description of Related Art

Conventional non-invasive blood glucose monitoring devices use rigid materials as substrates thereof, such as ceramic substrates, silicon substrates, semiconductor substrates, etc., and light emitting members and light sensing members are further disposed on the substrates to perform corresponding blood glucose monitoring functions. However, the rigid substrates tend to cause a lot of inconveniences when the conventional non-invasive blood glucose monitoring devices are fixedly set on bodies of users. Therefore, in order to facilitate the fixation of the non-invasive blood glucose monitoring devices on the bodies of users, the aforementioned devices must be equipped with wearable members for the bodies of users, such as bracelets, fixing belts, etc., which makes the aforementioned devices expose to clothing of the users, thereby affecting privacy of users.

In light of this, it is really worthy of research and development to design a non-invasive blood glucose monitoring device that solves those above-mentioned problems.

### SUMMARY OF THE INVENTION

An objective of the present invention is to provide a non-invasive blood glucose monitoring device with a flexible circuit board as a substrate.

Another objective of the present invention is to provide a non-invasive blood glucose monitoring device with concealment.

To achieve the above mentioned objectives, the non-invasive blood glucose monitoring device comprises a flexible circuit board, at least one blood glucose monitoring module and an adhesive layer. Wherein the at least one blood glucose monitoring module is arranged on the flexible circuit board, and each blood glucose monitoring module includes a barrier structure, a light emitting element, a light sensing element and a transparent cover. Wherein the barrier structure has a first space and a second space isolated from each other, the light emitting element and the light sensing element are located in the first space and the second space respectively, and the transparent cover is combined with the barrier structure to enclose the first space and the second space. Wherein the adhesive layer is arranged on the flexible circuit board and located around the at least one blood glucose monitoring module.

In one embodiment of the present invention, the flexible circuit board includes a base layer, a first conductive layer, and a first protective layer, and the base layer has a first surface and a second surface, and wherein the first conductive layer is disposed on the first surface, and the first protective layer is disposed on the first conductive layer.

In one embodiment of the present invention, the flexible circuit board further includes a second conductive layer and a second protective layer, and wherein the second conductive layer is disposed on the second surface, and the second protective layer is disposed on the second conductive layer.

In one embodiment of the present invention, the flexible circuit board defines an installation area, the at least one blood glucose monitoring module is arranged on the installation area, and the adhesive layer is located outside the installation area and surrounds the at least one blood glucose monitoring module.

In one embodiment of the present invention, a height of the adhesive layer is not less than an overall height of each blood glucose monitoring module.

In one embodiment of the present invention, a quantity of at least one blood glucose monitoring module is plural, and wherein the blood glucose monitoring modules are arranged in pairs at intervals on the flexible circuit board.

In one embodiment of the present invention, each of the at least one blood glucose monitoring module further includes a ceramic substrate, and each of the at least one blood glucose monitoring module is connected to the flexible circuit board through the ceramic substrate.

In one embodiment of the present invention, the barrier structure is made of non-transparent materials.

In one embodiment of the present invention, the first space and the second space is filled with a transparent packaging material for protecting the light emitting element and the light sensing element.

In one embodiment of the present invention, the transparent cover has a coating film, and the coating film is a complex layered structure formed by alternately stacking of silicon monohydride layers (SiH) and silicon dioxide layers.

Hereby, the non-invasive blood glucose monitoring device of the present invention, through disposing of the flexible circuit board with the adhesive layer, enables a monitoring surface of the blood glucose monitoring device to set on skins of users directly, and makes an installation surface relative to the monitoring surface stick inside of clothing of the users, duo to flexibility of the flexible circuit board. It provides an easy installation and a hidden effect for the device, compared with conventional non-invasive blood glucose monitoring devices.

### BRIEF DESCRIPTION OF THE DRAWINGS

FIG. 1 is a schematic view of a first embodiment of a non-invasive blood glucose monitoring device according to the present invention;

FIG. 2 is an overall top view of the first embodiment of the non-invasive blood glucose monitoring device according to the present invention;

FIG. 3 is a schematic view of a second embodiment of the non-invasive blood glucose monitoring device according to the present invention;

FIG. 4 is an overall top view of a third embodiment of the non-invasive blood glucose monitoring device according to the present invention;

FIG. 5 is a cross-sectional view along a section line A-A shown in FIG. 4;

FIG. 6 is a schematic view of a fourth embodiment of the non-invasive blood glucose monitoring device according to the present invention.

### DETAILED DESCRIPTION OF THE PREFERRED EMBODIMENT

Since various examples and embodiments in the present invention are only illustrative and non-restrictive, a person skilled in the art can easily conceive other examples and embodiments without contravening the scope of the present invention, after reading this specification, and can make the features and advantages of these embodiments more evident based on the following detailed description and claims.

Herein, the description of unit, element and component in the present invention uses “one”, “a”, or “an”. This is for convenience and for offering general meaning of the category of the present invention. Therefore, the description should be understood as including “one”, “at least one”, and singular and plural forms at the same time unless the context clearly indicates otherwise.

Herein, the description of the terms “first” or “second” and similar ordinal numbers are mainly used to distinguish or refer to the same or similar elements or structures and do not necessarily imply that such components or structures are spatially or temporally distinct order. It should be understood that ordinal numbers, in certain situations or configurations, may be used interchangeably without affecting the implementation of the present invention.

Herein, the description of “comprise”, “have” or other similar semantics have the non-exclusive meaning. For example, components or structures with a plurality of elements are not only limited to those disclosed in this specification, but also include generally inherent elements, which are not explicitly listed here for the components or the structures.

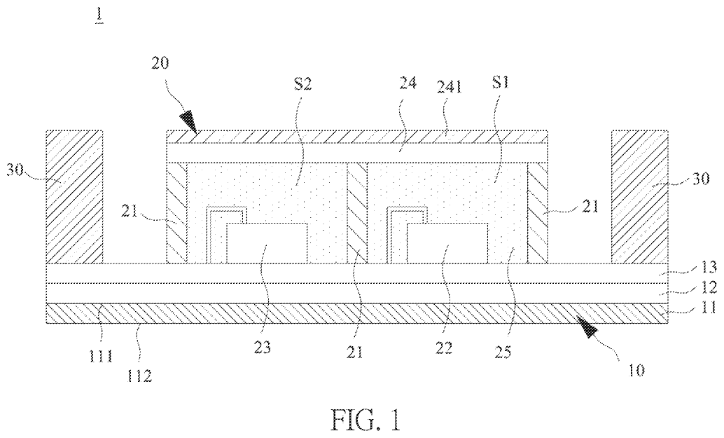

Please refer to FIG. 1, which is a schematic diagram of a first embodiment of a non-invasive blood glucose monitoring device of the present invention. As shown in FIG. 1, the non-invasive blood glucose monitoring device 1 of the present invention comprises a flexible circuit board 10, at least one blood glucose monitoring module 20 and an adhesive layer 30, wherein the at least one blood glucose monitoring module 20 and the adhesive layer 30 are arranged on the flexible circuit board 10. The flexible circuit board 10 is adopted to carry the at least one blood glucose monitoring module 20, the adhesive layer 30 and other related components, and the flexible circuit board 10 itself can also be electrically connected with the aforementioned components for signal transmission. In this embodiment, the flexible circuit board 10 at least includes a base layer 11, a first conductive layer 12 and a first protective layer 13. The base layer 11 is used as a main structural component, and the base layer 11 is made of polyimide (PI). The base layer 11 has a first surface 111 and a second surface 112 opposite to each other. The first conductive layer 12 is disposed on the first surface 111 of the base layer 11, and the first conductive layer 12 is made of copper to provide an electrical conduction effect. The first protective layer 13 is disposed on the first conductive layer 12, and the first protective layer 13 is made of transparent encapsulant materials to provide effects of insulation and protection. Herein, the flexible circuit board 10 is a single-layer flexible circuit board.

The at least one blood glucose monitoring module 20 is disposed on the flexible circuit board 10, and each blood glucose monitoring module 20 is electrically connected to the first conductive layer 12 of the flexible circuit board 10. In this embodiment, a quantity of the at least one blood glucose monitoring module 20 is one, but the location and quantity of the at least one blood glucose monitoring module 20 can be adjusted according to different design requirements. Each blood glucose monitoring module 20 includes a barrier structure 21, a light emitting member 22, a light sensing member 23 and a transparent cover 24. The barrier structure 21 is disposed on the flexible circuit board 10, and the barrier structure 21 forms a first space S1 and a second space S2 isolated from each other. In one embodiment of the present invention, the barrier structure 21 is made of non-transparent (for visible light and invisible light) materials, such as black colloid materials or other opaque colloid materials. In view of the above, the barrier structure 21 is able to prevent mutual light-interfering between the first space S1 and the second space S2.

The light emitting element 22 is disposed on the flexible circuit board 10, and the light emitting element 22 is located in the first space S1 formed by the barrier structure 21. The light emitting element 22 mainly serves as a light source to emit light toward skins. In the present invention, a single LED light source is used for the light emitting element 22, but the type, location and quantity of the light emitting element 22 can be adjusted according to different design requirements. For example, the aforementioned light emitting element 22 may adopt an LED light source for emitting short-wave infrared (SWIR), with a spectral range between 700 nm and 2000 nm, but not limited thereto. The light emitting element 22 can be electrically connected to the first conductive layer 12 of the flexible circuit board 10 through metal wires.

The light sensing element 23 is disposed on the flexible circuit board 10, and the light sensing element 23 is located in the second space S2 formed by the barrier structure 21. The light sensing element 23 mainly receives light back from the skins by diffuse reflection. In the present invention, one photo detector is used for the light sensing element 23, but the type, location and quantity of the light sensing element 23 can be adjusted according to different design requirements. For example, the light sensing element 23 may adopt a photo detector made of indium gallium arsenide (InGaAs), with a light absorption wavelength range between 900 nm and 1700 nm, but not limited thereto. The light sensing element 23 can also be electrically connected to the first conductive layer 12 of the flexible circuit board 10 through metal wires.

The transparent cover 24 is configured with the barrier structure 21, and openings of the first space S1 and the second space S2 are enclosed by combining of the transparent cover 24 and the barrier structure 21, so that the first space S1 and the second space S2 form enclosed spaces independent from each other, respectively. One side of the transparent cover 24 is used to connect to the barrier structure 21, and the opposite side is used to approach or contact subjects such as skins. The transparent cover 24 is made of transparent materials, such as glass, but not limited thereto. In addition, the transparent cover 24 has a coating film 241. The coating film 241 is formed on the side of the transparent cover 24 for approaching or contacting skins, and the structure and functional application of the coating film 241 can be adjusted according to different design requirements. For example, the coating film 241 can be a dual band pass filter film, which is mainly a complex layered structure formed by alternately stacking of silicon monohydride (SiH) layers and silicon dioxide (SiO2) layers, but not limited thereto. By disposing of the coating film 241, the transparent cover 24 can provide an effect of allowing light in a specific wavelength range to pass through, and effectively block stray light in other unnecessary wavelength ranges.

In the present invention, the first space S1 and the second space S2 can be filled with a transparent packaging material 25, such as liquid optical encapsulant or epoxy, but not limited thereto. The transparent packaging material 25 is able to provide an effect of auxiliary adhesion and fixing on the transparent cover 24, so as to enhance combining of the transparent cover 24 and the barrier structure 21. In addition, the transparent packaging material 25 is also able to provide a packaging effect for the light emitting element 22, the light sensing element 23 and the aforementioned metal wires and to provide an optical transmission property.

The adhesive layer 30 is disposed on the flexible circuit board 10, and the adhesive layer 30 is approximately located around the at least one blood glucose monitoring module 20. The adhesive layer 30 can be made of materials with elastic and skin-friendly properties, such as skin-friendly adhesive tape. Herein, the adhesive layer 30 may have a height, which is not less than the overall height of each blood glucose monitoring module 20 (such as a minimum distance counting from the surface of the first protective layer 13 of the flexible circuit board 10 to the transparent cover 24). For example, in this embodiment, the overall height of each blood glucose monitoring module 20 is about 1-2 mm, and the height of the adhesive layer 30 is also about 1-2 mm corresponding to each blood glucose monitoring module 20.

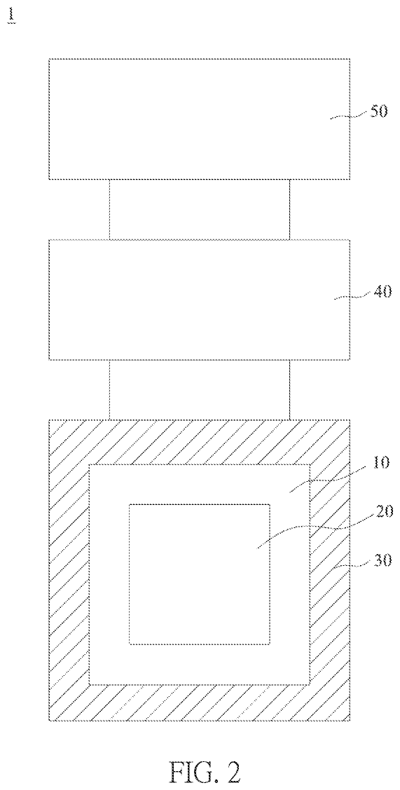

Please refer to FIG. 1 and FIG. 2 together, wherein FIG. 2 is an overall top view of the first embodiment of the non-invasive blood glucose monitoring device according to the present invention. As shown in FIG. 1 and FIG. 2, in the present invention, an installation area of the flexible circuit board 10 is larger than an area of the at least one blood glucose monitoring module 20. That is to say, the flexible circuit board 10 defines a sufficiently large installation area, and the at least one blood glucose monitoring module 20 can be arranged on the installation area of the flexible circuit board 10. For example, in this embodiment, the at least one blood glucose monitoring module 20 is disposed at a center of the flexible circuit board 10. The adhesive layer 30 is located outside the installation area and surrounds the at least one blood glucose monitoring module 20. And for example, in this embodiment, the adhesive layer 30 is disposed on the peripheral portion near the edge of the flexible circuit board 10.

Moreover, the non-invasive blood glucose monitoring device 1 of the present invention further comprises a driving unit 40 and a control unit 50. The driving unit 40 is electrically connected to the flexible circuit board 10 to drive the light emitting element 22 of each blood glucose monitoring module 20 to perform light emitting operation. Herein, the driving unit 40 is a LED driver board (client board). The control unit 50 is electrically connected to the driving unit 40 for sending signals to the driving unit 40 or receiving signals from the driving unit 40. Hereby, the control unit 50 is a circuit board (EVB or PCB).

When the non-invasive blood glucose monitoring device 1 of the present invention is in use, the transparent cover 24 of the at least one blood glucose monitoring module 20 can be attached to skins, and the flexible circuit board 10 around the at least one blood glucose monitoring module 20 can be adhered to the skins through the adhesive layer 30, so that a positioning effect or a fixation effect for the at least one blood glucose monitoring module 20 is provided. Due to the flexibility of the flexible circuit board 10, it is easy to fix the base layer 11 inside the clothing of users by sticking, sewing or clipping, and the drive unit 40 and the control unit 50 are also hidden inside the clothing. Accordingly, when the non-invasive blood glucose monitoring device 1 of the present invention is employed by a recipient, all components of the non-invasive blood glucose monitoring device 1 of the present invention can be completely hidden in the clothing of the recipient, so that it is difficult to be found by others and thus the privacy of the recipient is protected. At the same time, by replacing a conventional rigid material substrate by the flexible circuit board 10, its manufacturing cost is also effectively reduced.

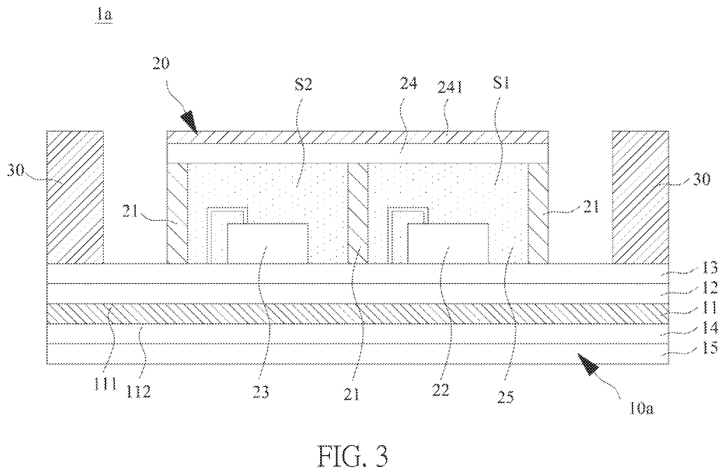

Please refer to FIG. 3, which is a schematic view of a second embodiment of the non-invasive blood glucose monitoring device according to the present invention. This embodiment is a modification of the aforementioned first embodiment, with a main point relative to the structure of the flexible circuit board. As shown in FIG. 3, in this embodiment, the flexible circuit board 10a of the non-invasive blood glucose monitoring device 1a of the present invention further comprises a second conductive layer 14 and a second protective layer 15. The second conductive layer 14 is disposed on the second surface 112 of the base layer 11, and the second conductive layer 14 is made of copper to provide a conductive effect. The second protective layer 15 is disposed on the second conductive layer 14, and the second protective layer 15 is made of transparent encapsulant materials to provide effects of insulation and protection. Herein, the flexible circuit board 10a is a double-layer flexible circuit board. Accordingly, the non-invasive blood glucose monitoring device 1a of the present invention is capable of providing faster and larger signal transmission functions through the flexible circuit board 10a.

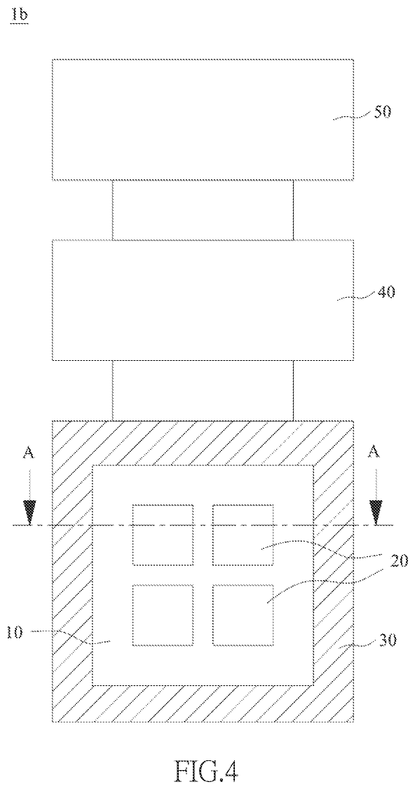

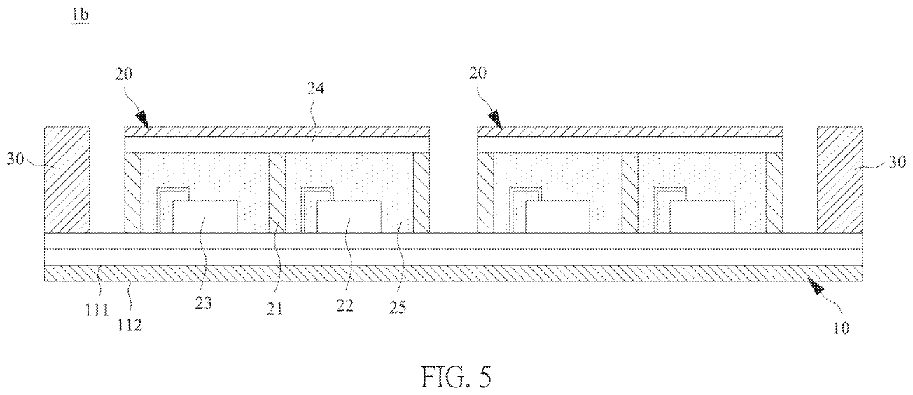

Please refer to FIG. 4 and FIG. 5 together, which are regarding the third embodiment of the non-invasive blood glucose monitoring device of the present invention. Wherein FIG. 4 is an overall top view of a third embodiment of the non-invasive blood glucose monitoring device according to the present invention, and FIG. 5 is a cross-sectional view along a section line A-A shown in FIG. 4. This embodiment is a modification of the aforementioned first embodiment, with a main point relative to a quantity of at least one blood glucose monitoring module 20. As shown in FIG. 4 and FIG. 5, in this embodiment, the quantity of the at least one blood glucose monitoring module 20 of the non-invasive blood glucose monitoring device 1b of the present invention is plural. These blood glucose monitoring modules 20 are separately arranged on the flexible circuit board 10. For example, the quantity of at least one blood glucose monitoring module 20 can be four, and these blood glucose monitoring modules 20 are arranged in pairs at intervals to form a 2×2 matrix of blood glucose monitoring modules. But the actual quantity can be increased or decreased according to the requirements. In view of the above, the non-invasive blood glucose monitoring device 1b of the present invention can simultaneously perform blood glucose monitoring operations through a plurality of blood glucose monitoring modules 20, thereby increasing accuracy and reliability of monitoring.

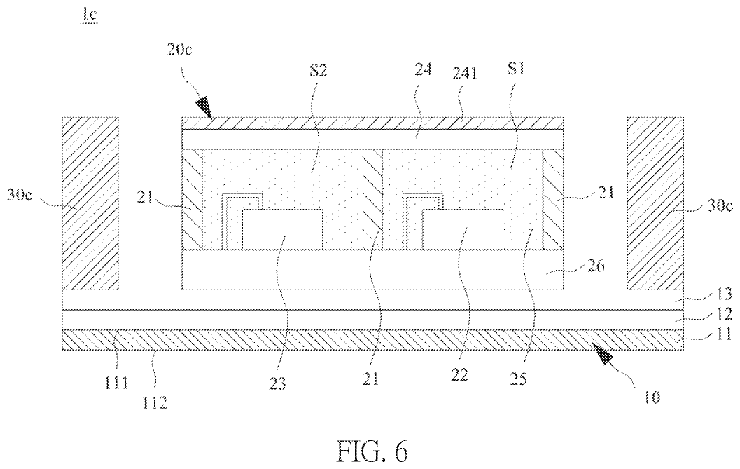

Please refer to FIG. 6, which is a schematic view of a fourth embodiment of the non-invasive blood glucose monitoring device according to the present invention. This embodiment is a modification of the first embodiment, with a main point relative to the structure of the at least one blood glucose monitoring module. As shown in FIG. 6, in this embodiment, each blood glucose monitoring module 20c of the non-invasive blood glucose monitoring device 1c of the present invention further includes a ceramic substrate 26, and each blood glucose monitoring module 20c is connected to the flexible circuit board 10 through the ceramic substrate 26. That is to say, by adopting a design of that a conventional blood glucose monitoring module with a ceramic substrate 26 matches with a flexible circuit board 10, each blood glucose monitoring module 20c provides a more stable monitoring result. In addition, in this embodiment, as a height of each blood glucose monitoring module 20c changes, a height of the adhesive layer 30c will also be adjusted accordingly.

It should be noted that any two or even more of the structures and technical features disclosed in all the above-mentioned embodiments of the non-invasive blood glucose monitoring device of the present invention can be combined according to requirements, so as to make the non-invasive blood glucose monitoring device of the present invention provide flexible applications under various conditions or states.

The above implementations are only auxiliary descriptions, and are not intended to limit the embodiments of the application subject or the applications or uses of the embodiments. In addition, although at least one illustrative example has been presented above, it should be understood that the present invention can still have a large number of variations. It should also be understood that the embodiments described herein are not intended to limit the scope, use, or configuration of the requested subject matter in any way. On the contrary, the foregoing embodiments will provide a convenient guide for those skilled in the art to implement one or more embodiments. Furthermore, various changes can be made to the function and arrangement of the components without departing from the scope defined by the patent claims, and the scope of the patent claims includes known equivalents and all foreseeable equivalents at the time that the patent application is filed.

What is claimed is:

1. A non-invasive blood glucose monitoring device, comprising:
a flexible circuit board,
at least one blood glucose monitoring module, arranged on the flexible circuit board, each blood glucose monitoring module including
a barrier structure, having a first space and a second space isolated from each other,
a light emitting element, located in the first space,
a light sensing element, located in the second space and
a transparent cover, combined with the barrier structure to enclose the first space and the second space, and
an adhesive layer, arranged on the flexible circuit board and located around the at least one blood glucose monitoring module.

2. The non-invasive blood glucose monitoring device as claimed in claim 1, wherein the flexible circuit board includes a base layer, a first conductive layer, and a first protective layer, and the base layer has a first surface and a second surface, and wherein the first conductive layer is disposed on the first surface, and the first protective layer is disposed on the first conductive layer.

3. The non-invasive blood glucose monitoring device as claimed in claim 2, wherein the flexible circuit board further includes a second conductive layer and a second protective layer, and wherein the second conductive layer is disposed on the second surface, and the second protective layer is disposed on the second conductive layer.

4. The non-invasive blood glucose monitoring device as claimed in claim 1, wherein the flexible circuit board defines an installation area, the at least one blood glucose monitoring module is arranged on the installation area, and the adhesive layer is located outside the installation area and surrounds the at least one blood glucose monitoring module.

5. The non-invasive blood glucose monitoring device as claimed in claim 1, wherein a height of the adhesive layer is not less than an overall height of each blood glucose monitoring module.

6. The non-invasive blood glucose monitoring device as claimed in claim 1, wherein a quantity of the at least one blood glucose monitoring module is plural, and wherein the blood glucose monitoring modules are arranged in pairs at intervals on the flexible circuit board.

7. The non-invasive blood glucose monitoring device as claimed in claim 1, wherein each of the at least one blood glucose monitoring module further includes a ceramic substrate, and each of the at least one blood glucose monitoring module is connected to the flexible circuit board through the ceramic substrate.

8. The non-invasive blood glucose monitoring device as claimed in claim 1, wherein the barrier structure is made of non-transparent materials.

9. The non-invasive blood glucose monitoring device as claimed in claim 1, wherein the first space and the second space is filled with a transparent packaging material for protecting the light emitting element and the light sensing element.

10. The non-invasive blood glucose monitoring device as claimed in claim 1, wherein the transparent cover has a coating film, and the coating film is a complex layered structure formed by alternately stacking of silicon monohydride layers and silicon dioxide layers.
